(19) United States
(12) Patent Application Publication
(10) Pub. No.: US 2022/0117220 A1
(43) Pub. Date: Apr. 21, 2022

(54) MINIMIZING IMMUNOGENICITY OF DECELLULARIZED TISSUES

(71) Applicant: TISSUE TESTING TECHNOLOGIES LLC, North Charleston, SC (US)

(72) Inventor: Kelvin GM BROCKBANK, Charleston, SC (US)

(73) Assignee: TISSUE TESTING TECHNOLOGIES LLC, North Charleston, SC (US)

(21) Appl. No.: 17/506,140

(22) Filed: Oct. 20, 2021

Related U.S. Application Data
(60) Provisional application No. 63/094,591, filed on Oct. 21, 2020.

(51) Int. Cl. A01N 1/02

(52) U.S. Cl. CPC A01N 1/0221

(57) ABSTRACT

A method for preserving and reducing the immunogenicity of a tissue, the method including obtaining a first tissue, the first tissue being a wild type tissue or genetically modified tissue; forming a second tissue by immersing the first tissue in a first solution having a cryoprotectant concentration of at least about 75% by weight for at least one hour to kill and lyse the cells of the first tissue; forming a third tissue by removing residual cell materials of the second tissue, the residual cell materials of the second tissue being removed by subjecting the second tissue to decellularization in a bioreactor; and subjecting the third tissue to ice-free cryopreservation.

### CROSS-REFERENCE TO RELATED APPLICATION

This nonprovisional application claims the benefit of U.S. Provisional Application No. 63/094,591 filed Oct. 21, 2020. The disclosure of the prior application is hereby incorporated by reference in its entirety.

### GOVERNMENT SUPPORT

The invention was supported, in whole or in part, by Grant #1R43AI114486-01A1 NIH, National Institute of Allergy and Infectious Diseases (Title: Immunogenicity of wild-type pig tissues after ice-free cryopreservation in genetically engineered recipients). The Government has certain rights in the invention.

### TECHNICAL FIELD

The present disclosure generally relates to methodology for preparing and/or preserving tissues for later use (e.g., for transplantation in the same or different mammals and/or for further research). In some embodiments, the present disclosure relates to simple straight forward methodology that enables tissues from a non-human source (such as porcine tissues) to be utilized in other mammals (such as humans) without chemical fixation or anti-α-Gal reactions. The present disclosure also provides a simple straight forward process that enables tissues from a non-human source (such as porcine donor tissues, for example where a galactose-α(1,3)-galactose antigen (α-Gal) epitope may or may not be initially present in the porcine donor tissue) to be utilized in humans and reduces recipient immune reactions (without chemical fixation or anti-α-Gal reactions).

### BACKGROUND

A number of implantable materials have been prepared from tissues originating from animals/non-humans (such as, for example, from porcine or bovine sources). Some of the available non-human replacement tissue options, such as xenogeneic porcine tissues for heart valve (HV) and ligament replacement, when unmodified, or wild-type (WT), have been known to initiate hyperacute transplant rejection and inflammation and subsequent structural deterioration (Mozzicato, 2014; Hawkins, 2016). Such problems are due, for example, to the presence of the galactose-α(1,3)-galactose antigen (α-Gal) epitope on the replacement tissues against which the recipient has pre-formed α-Gal antibodies. The complications associated with α-Gal are so severe that cardiovascular surgeons are even demanding that manufacturers of implantable xenogeneic transplantation materials address the issue of α-Gal in their products by providing α-Gal-free implantable materials, such as α-Gal-free heart valves (Ankersmit, 2017).

The presence of α-Gal is not limited to surface structures (and is a cause of bioprosthetic degeneration). Konakci (2005) demonstrated that α-Gal is contained within the connective tissue of bioprosthetic valves; Mozzicato (2014) described three patients with suspected α-Gal allergies, two of which developed immunologic reactions from porcine/bovine aortic valve replacement; and Hawkins (2016) observed premature bioprosthesis degeneration in two more patients after allergy development. It is thought that bioprosthetic valves undergo early calcification and degeneration due to the presence/distribution of α-Gal.

There is also a growing awareness in the art of the wide variety of potential triggers for α-Gal reactions in transplant recipients. These potential triggers may include administration of mammalian derived therapeutics (Cetuximab, heparin, gelatin capsules or hemostatic agents, colloids, vaccines, and HVs) or simply eating mammalian meat, such as beef, pork, lamb, etc. (Mullins, 2012; Chung, 2008; Platts-Mills, 2015; Steineke, 2015; van Nunen, 2018). An immune reaction precipitated by tick bites has also been linked to elevated α-Gal IgE titers that trigger anaphylaxis (termed α-Gal Syndrome (AGS)) after exposure via consumption of mammalian food (Van Nunen, 2015; Commins 2013) or implantation of medical products such as bioprosthetic HVs (Mozzicato, 2014; Hawkins, 2016)—up to 37% of the population in the Southeast United States have anti-α-Gal IgE titers that are considered allergen positive (Commins, 2009; 2011; Olafson 2014).

To address the potential α-Gal issues in implantable products, Mozzicato (2014) suggested that decellularized wild-type heart valves (WT HVs) could be considered for use in patients with IgE to α-Gal. However, it has been demonstrated that α-Gal is even bound to the extracellular matrix (ECM) and that it is chemically and heat stable. Therefore, decellularization alone is not effective (Takahashi, 2014; Mullins 2012, Apostolovic, 2014, 2016) to address the issue of α-Gal in implantable products. Furthermore, any decellularization strategy intense enough to remove α-Gal from WT pig tissues would need to break chemical bonds, and thereby degrade the ECM and compromising material properties.

In other strategies to address the potential α-Gal risks, the animal tissues used to form the implantable materials or to repair damaged tissues are chemically cross-linked with agents, such as glutaraldehyde, especially those animal tissue components that come into direct contact with the patient's blood. This sort of methodology represents the current standard of care with respect to implantable products/materials, such as bioprosthetic valves, and includes processing steps that are intended to reduce immunogenicity via “hiding” or “masking” antigens such as α-Gal. Such treatments are deemed to be necessary to prevent rejection of the implanted materials by the recipient (e.g., because many of the potential mammalian donor species (including, for example, New World monkeys, cows, pigs, mice, etc.,) express α-Gal on cells and tissue surfaces (Joziasse, 1989; Larsen, 1989; Sandrin, 1994)). While crosslinking the collagen matrix with agents such as glutaraldehyde may reduce antigenicity by “hiding” or “masking” antigens including α-Gal, unfortunately, such treatments (such as glutaraldehyde treatment) may obliterate the natural regenerative properties of the graft and residual α-Gal remains (Konakci, 2005; Bloch, 2011; Mangold, 2009; 2012).

Several different groups have also attempted to preserve the natural and regenerative properties of various xenograft tissues using milder tissue washing regimes and elimination of crosslinking with glutaraldehyde prior to transplantation. However, clinical results have ranged from catastrophic and lethal for HVs (Simon, 2003; Perri, 2012) to severe for vascular grafts (Sharp, 2004; Tolva, 2007) and for tendon augmentation for shoulder repair (Malcarney, 2005; Reider, 2005; Walton, 2007).

When processing steps were included that were intended to cleave or remove α-Gal (e.g. via galactosidase), the clinical results in a hernia model were improved (Xu, 2008; 2009; Sandor, 2008; Daly, 2009). However, removing the α-Gal epitope via galactosidase is, at best, a surface treatment—removing it from thick tissue matrices, particularly the ECM, has proven to be very difficult. Xenograft tendons have been attempted for restoration of the human ACL, past results predominately failed via inflammatory reaction or mechanical rupture (van Steensel, 1987). In an attempt in which the xenograft was processed with galactosidase and then with glutaraldehyde, there was a less vigorous immune response but inflammation persisted and grafts failed (Stone, 2007a; 2007b).

Thus, there remains a need for improved tissues for surgical replacement or repair of tissues that is not fulfilled by the above attempts. In particular, there remains a need for improvements using new, more effective processing methods and tissue sources that remove risks associated with the expression of the α-Gal epitope, particularly improvements in methodology that would significantly increase the number of non-human tissues that would be suitable for use as soft tissues for surgical replacement or repair of tissues.

### SUMMARY OF THE INVENTION

A method for preserving a tissue and reducing the immune reaction after transplantation or implantation of the preserved tissue, comprising: obtaining a first tissue from a donor, the first tissue being a wild type tissue or genetically modified tissue; forming a second tissue by immersing the first tissue in a first solution having a cryoprotectant concentration of at least about 75% by weight for at least one hour to kill and lyse the cells of the first tissue; forming a third tissue by removing residual cell materials of the second tissue, the residual cell materials of the second tissue being removed by subjecting the second tissue to decellularization in a bioreactor; subjecting the third tissue to ice-free cryopreservation, the ice-free cryopreservation including: infiltrating the third tissue with a second solution having a cryoprotectant concentration of at least about 75% by weight by placing the third tissue and the second solution in a container at a predetermined temperature for at least one hour, removing the second solution and replacing it with a third solution having a cryoprotectant concentration of at least about 75% by weight, sealing the container after the second solution has been replaced with the third solution such that the sealed container contains the third solution and third tissue, and storing the sealed container; and implanting or transplanting the third tissue into a recipient, wherein an immune reaction is not precipitated by the transplantation or implantation of the third tissue, or any immune reaction that occurs in the recipient is non-life-threating.

### BRIEF DESCRIPTION OF THE DRAWINGS

FIG. 1 (i.e., FIG. 1A and FIG. 1B) depicts illustrations of the data obtained with respect to an independent blind review of explant inflammation demonstrating that IFC vitrified α-Gal negative and WT explants from α-Gal negative recipients consistently have lower mean values than fresh tissue samples with many statistically significant differences; FIG. 1A shows the result with an aorta and FIG. 1A shows the result with tendon tissue explants after 2 or 4 weeks in vivo. Red bars indicate p<0.05 by both t-test and 1 way ANOVA. Black bars indicate p<0.05 by t-test only. If not indicated there was no significant difference.

FIG. 2 (i.e., FIG. 2A, FIG. 2B and FIG. 2C) depicts illustrations of a HV bioreactor for decellularization; FIG. 2A is an illustration of a valve mounting system; FIG. 2B is an illustration of an overview of system indicating air chamber (1), compliance (2), media reservoir (3), air filter (4), one-way check valve (5), variable constriction valve (6), pressure transducers (7,9), and a camera (8); and FIG. 2C is an illustration of an assembled bioreactor.

### DETAILED DESCRIPTION OF EMBODIMENTS

The present disclosure generally relates to methodology for preparing and/or preserving tissues for later use, such as for transplantation in the same or different mammals and/or for further research purposes.

More specifically, in some embodiments of the present disclosure tissues from wild type and/or genetically modified pigs, such as the Gal-safe pigs, may be dissected and antibiotic treated (e.g., by known methods). Examples of such tissues include heart valves, pericardium, blood vessels, ligaments, tendons, bladder, intestine, and skin. Then, the tissues will then be placed in a solution at a predetermined temperature (such as a VS83 solution at room temperature) for a predetermined amount of time sufficient to kill and lyse the cells in the tissue (such as for at least about 30 minutes, for at least about 60 minutes, for at least about 120 minutes, or for at least about 180 minutes to kill substantially all (e.g., greater than 90%) of the living cells present or all living cells present (e.g., by exposure to the extreme conditions, such as severe osmotic stresses and/or chemical cytotoxicity)).

In embodiments, the solution used to kill and lyse the cells in the tissue may contain about 75% to about 99% w/v cryoprotectant. For example, the solution used to kill and lyse the cells may contain dimethyl sulfoxide (DMSO), formamide, and 1,2 propanediol in a vehicle solution, such as Euro-Collins solution. Such a solution may contain about 75% to about 99% w/v dimethyl sulfoxide (DMSO), formamide, and 1,2 propanediol. The amount of dimethyl sulfoxide may be varied from 20 to 50% w/v. Similarly, the amount of 1,2 propanediol and formamide may each be varied from about 10 to 40% w/v. However, the total amount of cryoprotectant in the full strength solution (or final solution in which the tissue is placed in and/or infiltrated with) should be about 75 wt % or more of cryoprotectant, such as about 80% to about 99% cryoprotectant, or about 83% to about 95% cryoprotectant. Generally, the molarity of the cryoprotectant solution should be greater than about 6M (6 moles of cryoprotectant per liter of solution) for larger molecular weight cryoprotectants and higher for lower molecular weight cryoprotectants, such as, for example, a concentration of cryoprotectant from about 8M to about 25M, or a concentration of cryoprotectant from about 10M to about 20M, or a concentration of cryoprotectant from about 12M to about 16M.

In some embodiments, the tissue will be infiltrated with 83% CPA solution (such as VS83) containing 4.65 M DMSO, 4.65 M formamide, and 3.31 M 1,2 propanediol [propylene glycol (PG)] in Euro-Collins (EC) solution in one step by placing the tissues in sterile packaging along with, for example, 10-80 mL of VS83 in EC solution depending upon tissue volume, at a predetermined temperature (such as room temperature) on a shaker for a predetermined amount of time (such as those discussed above) sufficient to kill and lyse the cells in the tissue, such as for at least one hour.

Then the cell materials will be removed by washing in bioreactors under physiologic flow and pressure conditions using a sterile technique to remove residual cell materials. The bioreactors used may be any suitable bioreactor that is autoclaved, pre-washed and filled with sterile buffer solution (such as phosphate-buffered saline (PBS) with antibiotics at a suitable temperature, such as at about 37° C. Tissues will be treated for a predetermined amount of time, such as a treatment time of 0.5 to 10 days, such as about 1 to about 5 days, or a treatment time of 2 to 8 days, such as about 3 to about 6 days, or longer. Then, the tissues can be cryopreserved in a suitable preservation solution, such as in VS83.

The cryopreservation may be ice-free cryopreservation, such as ice-free cryopreservation performed by infiltrating with a suitable cryopreservation solution, such as a 83% CPA solution containing 4.65 M DMSO, 4.65 M formamide, and 3.31 M 1,2 propanediol [propylene glycol (PG)] in Euro-Collins (EC) solution in one step by placing the tissues in sterile polyester bags or vials at room temperature on a shaker for at least one hour. Then, the cryopreservation solution may be removed and replaced with fresh VS83 CPA solution, the bag heat sealed, and cooled for storage at any refrigerated temperature up to and including room temperature.

Such methodology of the present disclosure permits tissues from a non-human source (such as, for example, a source of donor tissues where a galactose-α(1,3)-galactose antigen (α-Gal) epitope may or may not be present in the donor tissue) to be utilized in humans and reduces recipient immune reactions (such as anti-α-Gal reactions), optionally without conventional chemical fixation processing steps intended to hide or mask antigens.

In embodiments, the decellularization may comprise detergent-free bioreactor decellularization methods and/or a combination of chemically induced osmotic cell destruction and dynamic bioreactor removal of cell debris.

One of the preferred embodiments of the present disclosure (for the preparation of the tissue products of the present disclosure, such as xenografts) combines donor tissues where a galactose-α(1,3)-galactose antigen (α-Gal) epitope is not present in the donor tissue (e.g., GalSafe® tissues) with an ice-free cryopreservation formulation that reduces recipient immune reactions and modulates tissue regeneration in combination with detergent-free decellularization in a dynamic flow bioreactor.

The above mentioned methodology of the present disclosure significantly decreases and/eliminates immunological responses to treated allogeneic tissue. That is, the methodology of the present disclosure is focused on reducing the tissue immune response in vitro and in vivo after elimination of the α-Gal epitope from consideration, by use of donor tissues where a galactose-α(1,3)-galactose antigen (α-Gal) epitope is not present in the donor tissue (e.g., GalSafe® tissues), followed by decellularization and ice-free cryopreservation.

Here, both human and “processed” xenograft scaffolds are proinflammatory when their cells are disrupted and cellular debris, cytokines, and other inflammatory moieties are not thoroughly removed from the ECM. Rieder et al. (2005) demonstrated that the lowest level of stimulation was with thoroughly “decellularized” human tissues. Decellularized porcine leaflets stimulated greater macrophage responses than extracts of human native pulmonary cusps that had not been decellularized. This observation, combined with the poor performance of decellularized porcine xenografts in patients (Simon, 2003) led to the selection of human allograft tissues—not xenografts—as the tissue source for decellularization technologies. The major US allograft HV processors, LifeNet Health and CryoLife, are focusing on development of decellularization methods and clinical experience is accumulating.

Conventional tissue processors either freeze with cryoprotective agents (CPAs), freeze without CPAs, decellularize or combine freezing methods with decellularization. In contrast, during recent years the inventors of the subject matter of the instant application have identified a CPA formulation (83%) that reduces tissue immunogenicity in vitro and in vivo. The various studies of the inventors combine to suggest mechanisms of action for CPA-induced modification of tissue immunogenicity.

From these studies and additional research the inventors of the subject matter of the instant application figured out that tissue treatment with a cryoprotectant solution, such as VS83, can be an alternative to detergent-based decellularization for allografts and that the two methods should be combined for donor tissues where a galactose-α(1,3)-galactose antigen (α-Gal) epitope is not present in the donor tissue, e.g., GalSafe® tissues, which led the inventors to assess the inflammatory response, remodeling, and immunogenicity of porcine tissues post-ice-free cryopreservation.

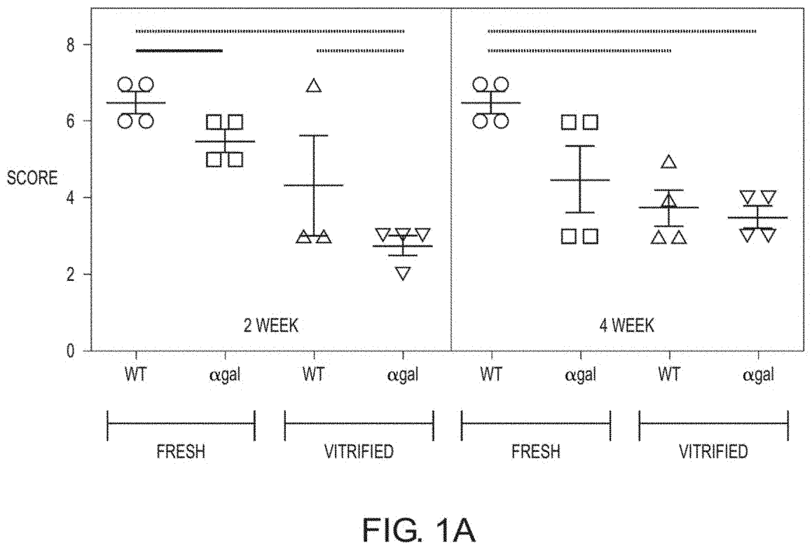

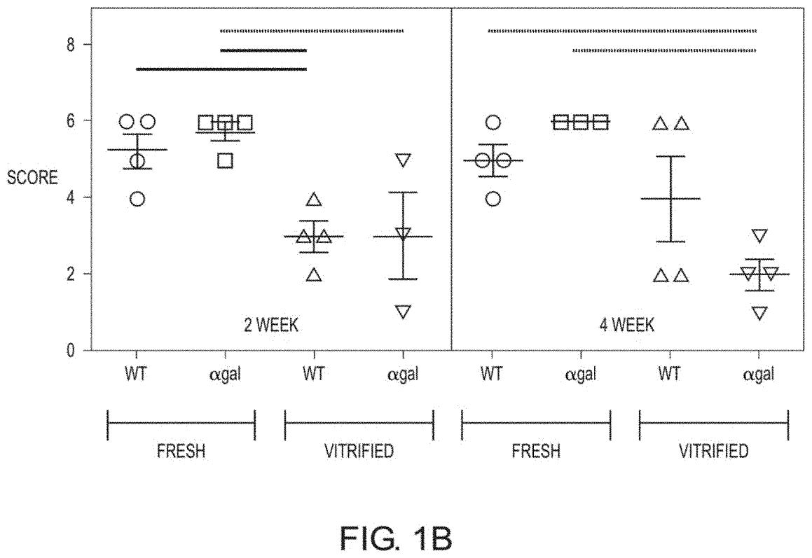

The inventors discovered that significantly less inflammation was observed in ice-free cryopreserved explants (Vitrified-WT and Vitrified-GalSafe® tissues), both aorta and tendon (as illustrated in FIG. 1). FIGS. 1A and 1B show an independent blind review of explant inflammation demonstrating that IFC vitrified α-Gal negative and WT explants from α-Gal negative recipients consistently have lower mean values than fresh tissue samples with many statistically significant differences. Aorta (FIG. 1A) and tendon (FIG. 1B) tissue explants after 2 or 4 weeks in vivo. Red bars indicate p<0.05 by both t-test and 1 way ANOVA. Black bars indicate p<0.05 by t-test only. If not indicated there was no significant difference.

The combination of ice-free cryopreservation and GalSafe®-derived aorta also resulted in significant differences in inflammatory cell frequency versus WT aortic explants.

Thus, a significant innovation regarding the methodology of the present disclosure (for the preparation of tissues for later use, such as xenografts) is the concept of combining donor tissues (such as those where a galactose-α(1,3)-galactose antigen (α-Gal) epitope is not present in the donor tissue (e.g., GalSafe® tissues)) with an ice-free cryopreservation formulation that reduces recipient immune reactions and modulates tissue regeneration in combination with detergent-free decellularization in a dynamic flow bioreactor.

In some embodiments, the raw tissues/materials (e.g., for fabrication into acellular scaffolds—tissue grafts without viable cells) for use with the decellularization and the ice-free tissue preservation methodologies of the present disclosure may be a tissue harvested from an animal that has been engineered and/or genetically modified, such as an animal lacking any expression of functional alpha-1,3-galactosyltransferase.

While there are certain mammals, such as catarrhines (humans, apes, and Old World monkeys), that do not have a functional GGTA1 gene and correspondingly do not express α-Gal that may be used in combination with the methodology of the present disclosure, a preferred application of the decellularization and ice-free tissue preservation methodology of the present disclosure is to engineered biological tissues, such as tissues harvested from a genetically engineered (GE) pig that has both alleles of GGTA1 inactive (α-Gal KO) and α-Gal is not detectable in these GE pigs. These engineered tissues may, for example, be obtained from any known source, such as from Revivicor Inc., which has developed a line of α-Gal KO, GalSafe®, pigs that is phenotypically normal (Liang, 2011; Fischer, 2012) compared with non-engineered wild-type (WT) pigs except for their genetically engineered trait. Such GalSafe® pigs may produce IgM and IgG against α-Gal (Fang, 2012).

In some embodiments, the genetically engineered (GE) pig may have both alleles of GGTA1 inactive (α-Gal KO) and α-Gal is not detectable (Dai, 2002; Phelps, 2003). Revivicor has demonstrated safety and efficacy by essentially completing all necessary steps for regulatory approval of the GalSafe® pig via the Food and Drug Administration Center for Veterinary Medicine (FDA, 2015). The intended use of the GalSafe® pigs is as a source of various raw materials for fabrication into acellular scaffolds (tissue grafts without viable cells) for further use (such as distribution as implantable human use medical products) or further testing. Any tissue derived from the GalSafe® pig including HVs, pericardium, vascular conduits, blood vessels, ligaments, tendons, bladder, intestine, skin and other tissues and organs, such as heart, may serve as materials for use in the methodology of the present disclosure.

Other animals that may or may not have been engineered and/or genetically modified may also be used in the methods of the present disclosure, such as an animal lacking any expression of functional alpha-1,3-galactosyltransferase, which are known to those of ordinary skill in the art, and are described, for example, in U.S. Pat. No. 7,795,493; U.S. application Ser. Nos. 10/646,970, 11/083,393, 12/835,026, 13/334,194, 14/281,464, 14/449,969, 15/905,249, 16/169,180; and WO2014066505A1, the entire enclosures of which are hereby incorporated by reference in their entireties.

Briefly, the animal donor source can be any animal, such as a ruminant or an ungulate, such as a bovine, porcine or ovine. In some embodiments, the animal is a pig. The raw materials/tissues from animals lacking any functional expression of the GGTA1 gene can be obtained from a prenatal, neonatal, immature, or fully mature animal, such as a porcine, bovine or ovine.

In embodiments, the raw materials/tissues for fabrication into acellular scaffolds (tissue grafts without viable cells) for use with the decellularization and the ice-free tissue preservation methodologies of the present disclosure are those harvested from an animal in which the alleles of the GGTA1 gene are rendered inactive, such that the resultant GGTA1 enzyme can no longer generate galactose alpha1,3-galactose (i.e., on the cell surface or elsewhere). For example, the raw materials/tissues for fabrication into acellular scaffolds (tissue grafts without viable cells) for use with the decellularization and the ice-free tissue preservation methodologies of the present disclosure may be those harvested from animals that lack any functional expression of alpha-1,3-galactosyltransferase, where the animal is selected from a porcine, a bovine and an ovine.

In some embodiments, the raw materials/tissues for fabrication into acellular scaffolds (tissue grafts without viable cells) for use with the decellularization and the ice-free tissue preservation methodologies of the present disclosure may be those harvested from animals in which one allele of the GGTA1 gene is inactivated via a genetic targeting event. In another aspect of the present disclosure, animals are provided in which both alleles of the GGTA1 gene are inactivated via a genetic targeting event. In some embodiments, the gene can be targeted via homologous recombination. In other embodiments, the gene can be disrupted, i.e. a portion of the genetic code can be altered, thereby affecting transcription and/or translation of that segment of the gene. For example, disruption of a gene can occur through substitution, deletion (“knockout”) or insertion (“knockin”) techniques. Additional genes for a desired protein or regulatory sequence that modulate transcription of an existing sequence can be inserted.

In some embodiments, the raw materials/tissues for fabrication into acellular scaffolds (tissue grafts without viable cells) for use with the decellularization and the ice-free tissue preservation methodologies of the present disclosure may be those harvested from animals in which the GGTA1 gene has been rendered inactive through at least one point mutation. In some embodiments, one allele of the GGTA1 gene can be rendered inactive through at least one point mutation. In embodiments, both alleles of the GGTA1 gene can be rendered inactive through at least one point mutation. In embodiments, this point mutation can occur via a genetic targeting event. In embodiments, this point mutation can be naturally occurring. In some embodiments, the point mutation can be a T-to-G mutation at the second base of exon 9 of the GGTA1 gene. In some embodiments, at least two, at least three, at least four, at least five, at least ten or at least twenty point mutations can exist to render the GGTA1 gene inactive.

The above-mentioned raw materials/tissues from animals lacking any functional expression of the GGTA1 gene can be obtained from a prenatal, neonatal, immature, or fully mature animal, such as a porcine, bovine or ovine. In some embodiments, the raw tissue can be subjected to further treatment or modification.

In some embodiments, the raw materials/tissues for fabrication into acellular scaffolds (tissue grafts without viable cells) for use with the decellularization and the ice-free tissue preservation methodologies of the present disclosure may be heart materials/tissues that are extracted from animals that lack any expression of the α-Gal epitope, which can be utilized without conventional chemical fixation processing steps intended to hide or mask antigens (more than half the heart valve market presently utilizes chemically cross-linked porcine and bovine tissues). For example, bovine, ovine, or porcine hearts, from animals lacking any functional expression of the α-Gal epitope, can serve as sources of heart materials/tissues. Types of such raw heart materials/tissues include, for example, the mitral valve, the aortic valve (also known as the atrial valve), the tricuspid valve, pulmonary valve, pulmonic patch, descending thoracic aorta, aortic non-valve conduit, pulmonic non-valve conduit with LPA and RPA, right or left pulmonary hemi-artery with or without intact cusp, saphenous vein, aortoiliac, femoral vein, femoral artery and/or semi-lunar valve. A heart valve xenograft prepared in accordance with the present disclosure may have the general appearance of a native heart valve xenograft. The heart valve xenograft may also be valve segments, such as individual leaflets, each of which may be implanted into recipient heart. Alternatively, porcine pericardium can be used to form the heart valve xenografts of the present disclosure suitable for use in open heart surgery or for transcatheter valve implantation methods.

In the methods of the present disclosure, the above-mentioned raw materials/tissues may be subject to detergent-free bioreactor decellularization methods and/or a combination of chemically induced osmotic cell destruction and dynamic bioreactor removal of cell debris.

For example, the methods of the instant disclosure may comprise methodology that effectively kills and lyses the cells in the tissue, such as: immersing the raw (e.g., dissected and antibiotic treated) tissue in a solution having a cryoprotectant concentration of at least 75% by weight (i.e., a single solution) or immersing the decellularized tissue in a series of solutions with the final solution having a cryoprotectant concentration of at least 75% by weight; where the raw tissue is held for a predetermined amount of time sufficient to kill and lyse the cells in the raw tissue (such as for at least about 30 minutes, for at least about 60 minutes, for at least about 120 minutes, or for at least about 180 minutes to kill substantially all (e.g., greater than 90%) of the living cells present or all living cells present (e.g., by exposure to the extreme conditions, such as severe osmotic stresses and/or chemical cytotoxicity)). The solutions used to kill and lyse the cells may be the same as those discussed below in regards to the tissue ice-free cryopreservation (IFC) with the caveat that the molecules must be able to effectively permeate the raw tissues (e.g., non-penetrating chemicals and/or impermeable cryoprotectant agents such as polyvinylpyrrolidone or hydroxyethyl starch may not be effective due to size restrictions that preclude some of the high molecular weight CPAs from being effectively used in this regard).

In some embodiments, a majority, substantially all (e.g., greater than 90%), or all of the cells of the raw tissue may be killed by manipulation of the magnitude of the step up in cryoprotectant concentration by the use of a single, stepwise, or gradient increase in cryoprotectant concentration. The cytotoxicity of the cryoprotectant solution may also kill the cells of the tissue. The cytotoxicity of the cryoprotectant solution increases as tissue (and solution) temperatures closer to 37° C. are achieved. In embodiments, exposing the tissue to the cryoprotectant at such temperatures may kill a majority, substantially all (e.g., greater than 90%), or all of the cells of the tissue because of the increased level of cytotoxicity of the cryoprotectant solution. In embodiments, the temperature at which the tissue may be held and exposed to cryoprotectants and/or the solution temperature at which the single, stepwise, or gradient increase in cryoprotectant concentration occurs to carry out the killing/lysing of the cells of the tissue may be in the range of from about 0° C. to about 37° C., such as about 10° C. to about 37° C., or about 25° C. to about 37° C. The duration that the tissue may be immersed in such solution having an increased cryoprotectant concentration will be a function of the mass of the raw tissue, and may be at least about 30 minutes, for at least about 60 minutes, for at least about 120 minutes, or for at least about 180 minutes, or in the range of from 30 minutes to 600 minutes, in the range of from 60 minutes to 300 minutes, or in the range of from 90 minutes to 150 minutes.

The volume of the solutions employed may vary considerably, such as from about 1 to about 100 milliliters (mL) or greater of necessary, or from about 10 to about 80 mL, or from about 10 to about 40 mL, or from about 15 to about 25 mL, based upon the size of the tissue being immersed in solution.

Then, in embodiments, these raw tissues in which all or substantially all (e.g., greater than 90%) of the living cells have been killed or lysed may be placed in bioreactors under physiologic flow and pressure conditions using sterile technique. The bioreactors may be autoclaved, pre-washed and filled with sterile agents, such as PBS with antibiotics (for example, at 37° C.).

The methods of the instant disclosure may also comprise an ice-free cryopreservation (IFC) process that comprises: immersing the decellularized tissue (where the above-mentioned raw tissue has been decellularized) in a solution having a cryoprotectant concentration of at least 75% by weight or immersing the decellularized tissue in a series of solutions with the final solution having a cryoprotectant concentration of at least 75% by weight; and single, gradient, or stepwise cooling step, wherein the decellularized tissue is cooled to a temperature between below the glass transition temperature of the first solution and −20° C.; a storage step, wherein the decellularized tissue is stored at temperature between below the glass transition temperature of the cryoprotectant solution and −20° C.; an optional rewarming step, wherein the decellularized tissue is warmed in a single, gradient, or stepwise rewarming step; and an immersion or washing step occurring during or after the rewarming step, where the cryoprotectant is washed out of the decellularized tissue in a single, gradient or multiple steps.

The simplicity, versatility, and scalability of methodology of the present disclosure allow for cost effective long-term storage and shipping, rapid clinical translation and market penetration for treated products. Such methodology of the present disclosure can have far-reaching clinical impact on surgical repairs by providing unprecedented access to low cost tissues with retention of structural and mechanical properties while minimizing immunogenicity.

While the discussion below mainly identifies xenogeneic porcine tissues (harvested from a GE pig that has both alleles of GGTA1 inactive (α-Gal KO) and α-Gal is not detectable in these GE pigs) as the tissues being acted upon by the methodology of the present disclosure, other organs and tissues, such as, for example, harvested from other GE mammals, or even other certain mammals, such as catarrhines (humans, apes, and Old World monkeys), that do not have a functional GGTA1 gene and correspondingly do not express α-Gal, may also be used with the methodology of the present disclosure.

In embodiments, the disclosure provides a simple straight forward process that allows porcine tissues harvested from a GE pig to be utilized in humans and reduces recipient immune reactions (e.g., without chemical fixation and/or anti-α-Gal reactions). For example, in some embodiments, the methodology of the present disclosure applies ice-free cryopreservation (IFC) technology to α-Gal knockout pig tissues (harvested from genetically modified GalSafe® pigs) to arrive at suitable scaffolds for tissue replacement without chemical fixation.

The benefits of combining IFC methodology with GalSafe® tissues affords improved xenogeneic tissue methods that result in a tissue product that is significantly less immunogenic, particularly when combined with detergent-free decellularization to remove residual cell materials (e.g., in the production of xenograft heart valve products). In such embodiments, engineered biological tissues, such as those obtained from GalSafe® pigs, may be used as a source of various raw materials for fabrication into acellular scaffolds (tissue grafts without viable cells) for use with the decellularization and the ice-free tissue preservation methodologies of the present disclosure.

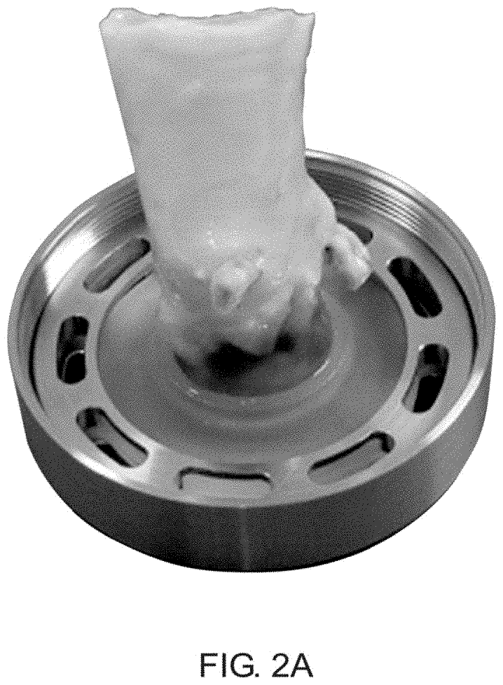

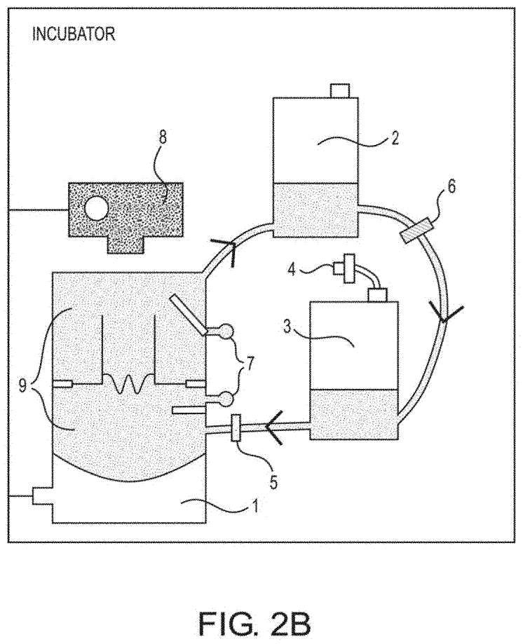

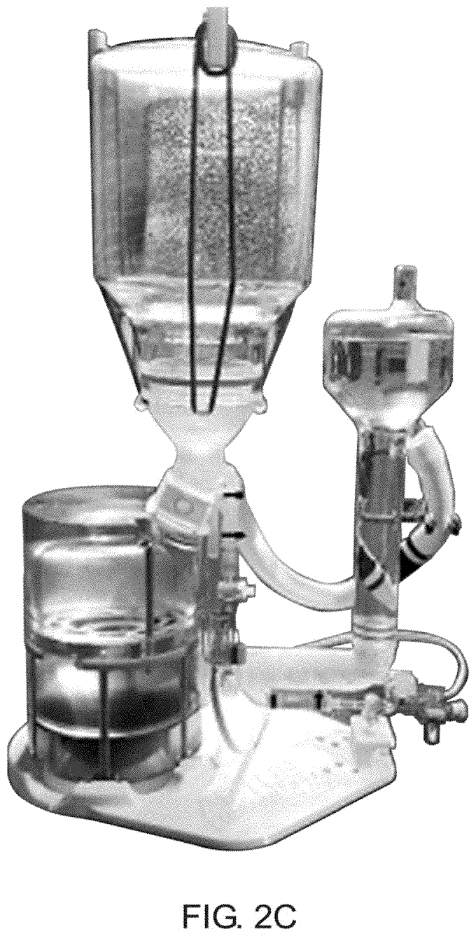

In embodiments, any suitable bioreactor known to those of ordinary skill in the art may be used. For example, in embodiments, the HV bioreactor illustrated in FIG. 2 may be used (developed by Tedder, 2003; 2009; Sierad, 2010). That is an HV bioreactor shown as depicted in FIGS. 2A-C, or a similar bioreactor may be used for decellularization. FIG. 2A shows an exemplary valve mounting system. FIG. 2B shows an overview of system indicating air chamber (1), compliance (2), media reservoir (3), air filter (4), one-way check valve (5), variable constriction valve (6), pressure transducers (7,9), and a camera (8). And, FIG. 2C shows an assembled bioreactor.

Such a bioreactor is powered by an external air pump, the diaphragm bulges into the pumping chamber and pushes the residing media through the HV into the chamber. Once a cycle is complete, the pump releases pressure in the air chamber, thereby allowing the hydrostatic pressure of the media to push the membrane down and create lower pressure in the pumping chamber to close the HV. All chambers have multiple ports for easy access of pressure transducers, media sampling and other probes. One-way valves ensure unidirectional flow of media. The bioreactor may contain about 800 mL of liquid and produces physiologic pulsatile flows at systemic pressures and variable stroke rates. Up to 4 bioreactors can be run in one standard-size cell culture incubator. The clear, flat top of the aortic chamber facilitates unobstructed recording of leaflet motions using a camera that allows uninterrupted remote monitoring of the HV from any digital device. The system continuously measures and controls frequency (bpm), open/closed time (duty cycle), stroke volume (flow rate), systolic/diastolic pressures, viscosity, temperature and gases, if needed. The bioreactor may be operated such that it can be maintained sterile for up to 4 weeks with weekly manual media changes. Such a bioreactor allows full control of physiologic pulmonary or aortic valve conditions. In embodiments involving such a bioreactor, HVs may be exposed to physiologic pressure and flow conditions for 1-7 days for decellularization, or longer if needed, for example to achieve a target of greater than 99% DNA free (which may be assessed via DNA analysis, tissue MALDI, proteomics and AGS patient antibody binding studies (Apostolovic, 2014). In some embodiments, the decellularized tissue may be substantially free of nucleic acids, such as 90, 90, 91, 92, 93, 94, 95, 96, 97, 98, 99, or 100% free of nucleic acids. In some embodiments, the nucleic acid is RNA. In some embodiments, the nucleic acid is DNA. In some embodiments, the tissue has less than 0.5 ng/mg (dry weight of tissue) of DNA. In some embodiments, the decellularized tissue has less than 0.5 ng/mg (dry weight of tissue) of DNA as measured by any suitable assay, such as a PicoGreen® DNA Assay.

The heart valve xenograft of the present disclosure, or a segment thereof, can be implanted into damaged human or animal hearts by those of skill in the art using known surgical techniques, for example, by open heart surgery, or minimally invasive techniques such as transcatheter endoscopic surgery, and transluminal implantation. Specific instruments for performing such surgical techniques are known to those of skill in the art, which ensure accurate and reproducible placement of heart valve implants.

In some embodiments, decellularization processes used in the present disclosure may be detergent free. In embodiments, the detergent-free decellularization may include physically-based processes, a chemically-based process, or biochemically-based processes provided that the donor tissue is rendered acellular or substantially acellular (i.e., greater than 99% DNA free). If necessary, decellularization may also be accomplished using a number of chemical treatments, including incubation in certain salts, or enzymes.

In some embodiments, protease inhibitors (such as phenylmethanesulfonylfluoride (PMSF), aprotinin, leupeptin, and Ethylenediamineteteraacetic acid (EDTA)) may be employed (for example, before during or after the donor tissue is subjected to detergent-free decellularization in a dynamic flow bioreactor) in combination with other reagents to prevent degradation of the extracellular matrix. Collagen-based connective tissues contain proteases and collagenases as endogenous enzymes in the extracellular protein matrix. Additionally, certain cell types including smooth muscle cells, fibroblasts and endothelial cells contain a number of these enzymes inside vesicles called lysosomes. When these cells are damaged by events such as hypoxia, the lysosomes are ruptured and their contents released. As a result, the extracellular matrix can undergo severe damage from protein, proteoglycan and collagen breakdown. This damage can be severe, as evidenced in clinical cases of cardiac ischemia where a reduction in oxygen which is insufficient to cause cell death results in pronounced damage to the collagen matrix. Additionally, a consequence of extracellular breakdown is the release of chemoattractants, which solicit inflammatory cells, including polymorphonuclear leukocytes and macrophages, to the graft, which are intended to remove dead or damaged tissue. These cells also, however, perpetuate the extracellular matrix destruction through a nonspecific inflammatory response. Accordingly, the methodology of the present disclosure may contain one or more protease inhibitors selected from the group of N-ethylmaleimide (NEM), phenylmethylsulfonylfluoride (PMSF) ethylenediamine tetraacetic acid (EDTA), ethylene glycol-bis-(2-aminoethyl(ether)NNN′N′-tetraacetic acid, ammonium chloride, elevated pH, apoprotinin and leupeptin to prevent such damage.

In some embodiments, a combination of physical treatments and chemical or biochemical treatments can be used for tissue decellularization in the bioreactor. For example, cells may be lysed physically using osmotic gradients, mechanical compression/massage, or freeze-thaw cycles. Hypertonic and hypotonic treatments put hydrostatic pressure on cell membranes in an aqueous environment causing them to burst and release their cell contents. The contents of the cell are then more accessible to later treatments with isotonic washout procedures. Mechanical compression or massage can be used to encourage membrane degradation and gradually expose more cell membranes to extraction solutions. If necessary, freeze-thaw cycles may used to kill cells and then fracture their cell membranes so that subsequent washout procedures can access internal cell contents and fragmented membranes.

In embodiments, decellularization processes used in the present disclosure may be detergent free. Detergents are chemicals that when they are added in a sufficient concentration to form micelles. A micelle is a cluster of detergent monomers, often spherical, that is oriented so that the non-polar domains of the detergent molecules are interacting internally, and the polar domains are interacting with water molecules externally. Detergents can be classified by one of three designations: ionic, nonionic, and zwitterionic. Ionic detergents are either anionic or cationic. A subgroup of ionic detergents are the bile acid salts, found in the intestine where they solubilize fats. Nonionic detergents, such as Triton X-100®, have neutral polar head groups and are non-denaturing to proteins. Zwitterionic detergents, such as CHAPS, have properties of both ionic and nonionic detergents. Zwitterionic detergents are generally milder than ionic detergents and more denaturing to proteins than nonionic detergents.

Various solvents can be used according to the present methods for the detergent free decellularization processes used in the present disclosure. In this regard, any solvent showing good decellularization performance with very little damage to the extracellular matrix may be used.

The ice-free cryopreservation (IFC) methodology of the present disclosure for application to tissues or decellularized tissues (hereinafter collectively referred to as “tissue” or “tissues”) may comprise: immersing the tissue in a solution having a cryoprotectant concentration of at least 75% by weight or immersing the tissue in a series of solutions with the final solution having a cryoprotectant concentration of at least 75% by weight; and single, gradient, or stepwise cooling step, wherein the tissue is cooled to a temperature between the glass transition temperature of the first solution and −20° C.; a storage step, wherein the tissue is stored at temperature between the glass transition temperature of the cryoprotectant solution and −20° C.; an optional rewarming step, wherein the tissue is warmed in a single, gradient, or stepwise rewarming step; and an immersion or washing step occurring during or after the rewarming step, where the cryoprotectant is washed out of the tissue in a single, gradient or multiple steps.

“Tissue” is used herein to refer to any natural or engineered biological extracellular tissue matrices that do not require living, viable cells, including extracellular tissue matrices of vascularized tissues and avascular tissues, including vascular tissue, such as blood vessels, musculoskeletal tissue, such as cartilage, menisci, muscles, ligaments and tendons, skin, cardiovascular tissue, such as heart valves and myocardium, periodontal tissue, peripheral nerves, bladder, gastro-intestinal tract tissues, ureter and urethra. “Blood vessel” is used herein to refer to any biological tube conveying blood. Thus, the phrase refers, inter alia, to an artery, capillary, vein, sinus or engineered construct.

As used herein, the term “transplantation” refers to any type of transplantation or implantation whether or not autologous, homologous or heterologous, and whether or not it is performed directly or subsequent to further processing of the tissue.

As used herein, the term “non-life-threatening” refers to an immune reaction where there is a strong possibility (such as greater that 95% or greater than 99%) that the reaction or the complications/circumstances/situation related thereto will not kill them within a predetermined amount of time, such as within one month, or within 1 year or within 5 years.

As used herein, the term “vitrification” refers to solidification without ice crystal formation. As used herein, a tissue is vitrified when it reaches the glass transition temperature (Tg). The process of vitrification involves a marked increase in viscosity of the cryoprotectant solution as the temperature is lowered such that ice nucleation and growth are inhibited. In practice, vitrification or vitreous cryopreservation may be achieved even in the presence of a small, or restricted amount of ice, which is less than an amount that causes injury to the tissue.

As used herein, the “glass transition temperature” refers to the glass transition temperature of a solution or formulation under the conditions at which the process is being conducted. In general, the process of the present invention is conducted at physiological pressures. However, higher pressures can be used as long as the tissue is not significantly damaged thereby.

As used herein, “physiological pressures” refer to pressures that tissues undergo during normal function. The term “physiological pressures” thus includes normal atmospheric conditions, as well as the higher pressures that various tissues, such as vascularized tissues, undergo under diastolic and systolic conditions.

As used herein, the term “perfusion” means the flowing of a fluid through the tissue. Techniques for perfusing organs and tissues are discussed in, for example, U.S. Pat. No. 5,723,282 to Fahy et al., which is incorporated herein in its entirety.

As used herein, the term “cryoprotectant” means a chemical that minimizes ice crystal formation in a tissue when the tissue is cooled to subzero temperatures and results in substantially no damage to the tissue after warming, in comparison to the effect of cooling without cryoprotectant.

As used herein, the term “substantially cryoprotectant-free tissue” refers to a tissue having substantially no cryoprotectant therein, such as a tissue containing less than 2% by weight cryoprotectant, or a tissue having less than 1% by weight cryoprotectant, or a tissue having less than 0.1% by weight cryoprotectant. As used herein, the term “cryoprotectant-free tissue” refers to a tissue having no cryoprotectant therein.

As used herein, the term “substantially cryoprotectant-free solution” refers to a solution having substantially no cryoprotectant therein, such as a solution containing less than 1% cryoprotectant by weight, or a solution having less than 0.5% cryoprotectant by weight, or a solution having less than 0.1% cryoprotectant by weight. As used herein, the term “cryoprotectant-free solution” refers to a solution having no cryoprotectant therein.

As used herein, “approximate osmotic equilibration” means that there is no more than a 10% difference between the intracellular and extracellular solute concentrations, such as no more than a 5% difference between the intracellular and extracellular solute concentrations. A difference of no more than 10% means, for example, that, if the extracellular concentration is 4M, the intracellular solute concentration is between 3.6 and 4.4M.

Vitrification may be achieved using a variety of cryoprotectant mixtures and cooling/warming conditions. The key variables should be optimized for each particular extracellular tissue matrix type and sample size. The choice of cryoprotectant mixtures and the equilibration steps necessary for cryoprotectant addition and removal without undue osmotic shock should be optimized based upon measured kinetics of cryoprotectant permeation in tissue samples. Cryosubstitution can also be employed to verify that ice-free preservation has been achieved for a given protocol.

Embodiments may comprises a single or a stepwise cooling process, such as, when the tissue is cooled (at a constant rate) in a first solution containing cryoprotectant at temperature between the glass transition temperature of the first solution and −20° C.; and a storage step, wherein the tissue is stored at temperature between the glass transition temperature of the first solution and −20° C.

The single cooling step may also be performed in a single step of decreasing the temperature of the tissue where the rate of cooling stays constant, or changes by either increasing or decreasing. Alternatively, the tissue may be cooled in a stepwise cooling process in which the temperature of the tissue is decreased to a first temperature in a first solution containing cryoprotectant at a first temperature between the glass transition temperature of the first solution and −20° C., then is further decreased to a second temperature in a second solution containing cryoprotectant at temperature between the glass transition temperature of the first solution and −20° C., and this process may be repeated with a third, fourth, fifth, sixth, seventh, etc., solution until the desired temperature is achieved.

In embodiments, the glass transition temperature of the first solution (cryoprotectant solution formulation) is in the range from about −100° C. to about −140° C., such as about −110° C. to about −130° C., or −115° C. to about −130° C., for example about −124° C. In embodiments, the tissue may be cooled and subsequently stored at temperatures between the glass transition temperature and about −20° C., such as about −120° C. to about −20° C., such as between about −110° C. to about −30° C., or between about −90° C. and about −60° C.

During the cooling step and the storage step, it is important to prevent tissue-glass cracking and ice forming. In contrast to other cryopreservation methods, a method for preserving a tissue, such as mammalian tissue, is focused on matrix preservation alone, and the method need not be specifically designed to preserve cells in a viable state.

In embodiments, a single cooling step; a stepwise cooling process at either regular, increasing, or decreasing intervals; or a gradient cooling step in which the rate of cooling is increased or decreased during the cooling process, may be used to cool the tissue to a temperature in the range of about −60° C. to about −100° C., such as −70° C. to about −90° C., for example about −80° C.

By employing a high concentration cryopreservation solution formulation, cooling and storage at a temperature between the glass transition temperature of the cryoprotectant formulation and about −20° C. may be attained without tissue-glass cracking and ice nucleation. In embodiments, the first solution contains about 75 wt % or more of cryoprotectant, such as about 80% to about 99% cryoprotectant, or about 83% to about 95% cryoprotectant.

After being immersed in a cryoprotectant-free solution, the tissue may be immersed in a solution containing cryoprotectant with or without perfusion. The final cryoprotectant concentration may be reached in a stepwise cooling process in which the tissue may be immersed in a first solution containing a first cryoprotectant concentration, then the tissue may be immersed in a second solution containing a second cryoprotectant concentration (which is higher than the first cryoprotectant concentration), and this process may be repeated with a third, fourth, fifth, sixth, seventh, etc., solution until the desired concentration is achieved. The cryoprotectant solution may contain any combination of cryoprotectants. Cryoprotectants include, for example dimethyl sulfoxide, 1,2-propanediol, ethylene glycol, n-dimethyl formamide and 1,3-propanediol in addition to those listed below in Table 1.

TABLE 1

Acetamide
Agarose
Alginate
Alanine
Albumin
Ammonium acetate
Butanediol
Chondroitin sulfate
Chloroform
Choline
Cyclohexanediols
Dextrans
Diethylene glycol
Dimethyl acetamide
Dimethyl formamide
Dimethyl sulfoxide
Erythritol
Ethanol
Ethylene glycol
Ethylene glycol monomethyl ether
Formamide
Glucose
Glycerol
Glycerophosphate
Glyceryl monoacetate
Glycine
Hydroxyethyl starch
Inositol
Lactose
Magnesium chloride
Magnesium sulfate
Maltose
Mannitol
Mannose
Methanol
Methoxy propanediol
Methyl acetamide
Methyl formamide
Methyl ureas
Methyl glucose
Methyl glycerol
Phenol
Pluronic polyols
Polyethylene glycol
Polyvinylpyrrolidone
Proline
Propylene glycol
Propanediol
Pyridine N-oxide
Ribose
Serine
Sodium bromide
Sodium chloride
Sodium iodide
Sodium nitrate
Sodium nitrite
Sodium sulfate
Sorbitol
Sucrose
Trehalose
Triethylene glycol
Trimethylamine acetate
Urea
Valine
Xylose

Impermeable cryoprotectant agents such as polyvinylpyrrolidone or hydroxyethyl starch may be more effective at protecting biological systems cooled at rapid rates. Such agents are often large macromolecules, which affect the properties of the solution to a greater extent than would be expected from their osmotic pressure. Some of these non-permeating cryoprotectant agents have direct protective effects on the cell membrane. However, the primary mechanism of action appears to be the induction of extracellular glass formation. When such cryoprotectants are used in extremely high concentrations, ice formation may be eliminated entirely during cooling to and warming from cryogenic temperatures. Impermeable chemicals with demonstrated cryoprotective activity include agarose, dextrans, glucose, hydroxyethylstarch, inositol, lactose, methyl glucose, polyvinylpyrrolidone, sorbitol, sucrose and urea.

In embodiments, the cryoprotectant solution contains dimethyl sulfoxide, formamide, and 1,2-propanediol in a vehicle solution, such as Euro-Collins solution. Such a solution may contain about 75% to about 99% w/v cryoprotectant. The amount of dimethyl sulfoxide may be varied from 20 to 50% w/v. Similarly, the amount of 1,2-propanediol and formamide may each be varied from about 10 to 40% w/v. However, the total amount of cryoprotectant in the full strength solution (or final solution in which the tissue is stored) should be about 75 wt % or more of cryoprotectant, such as about 80% to about 99% cryoprotectant, or about 83% to about 95% cryoprotectant. The molarity of cryoprotectant in a 75 wt % or more of cryoprotectant solution (i.e., the final solution in which the tissue is stored) will depend on the molecular weight of the cryoprotectant. Generally, the molarity of the cryoprotectant solution should be greater than about 6M (6 moles of cryoprotectant per liter of solution) for larger molecular weight cryoprotectants and higher for lower molecular weight cryoprotectants, such as, for example, a concentration of cryoprotectant from about 8M to about 25M, or a concentration of cryoprotectant from about 10M to about 20M, or a concentration of cryoprotectant from about 12M to about 16M.

The cryoprotectant solution may also be modified with conventional cryoprotectants and/or natural or synthetic ice-blocking molecules, for example, acetamide, agarose, alginate, alanine, albumin, ammonium acetate, anti-freeze proteins, butanediol, chondroitin sulfate, chloroform, choline, cyclohexanediols, cyclohexanediones, cyclohexanetriols, dextrans, diethylene glycol, dimethyl acetamide, dimethyl formamide, erythritol, ethanol, ethylene glycol, ethylene glycol monomethyl ether, glucose, glycerol, glycerophosphate, glyceryl monoacetate, glycine, glycoproteins, hydroxyethyl starch, inositol, lactose, magnesium chloride, magnesium, sulfate, maltose, mannitol, mannose, methanol, methoxy propanediol, methyl acetamide, methyl formamide, methyl ureas, methyl glucose, methyl glycerol, phenol, pluronic polyols, polyethylene glycol, polyvinylpyrrolidone, proline, pyridine N-oxide, raffinose, ribose, serine, sodium bromide, sodium chloride, sodium iodide, sodium nitrate, sodium nitrite, sodium sulfate, sorbitol, sucrose, trehalose, triethylene glycol, trimethylamine acetate, urea, valine and/or xylose.

In addition, in further embodiments of the invention, 1,2-propanediol may be replaced by similar concentrations of 2,3-butanediol. Similarly, dimethyl sulfoxide may be replaced by similar concentrations of glycerol or ethylene glycol or combinations thereof.

In embodiments, the cryoprotectant solution formulation may contain at least one or more of cryoprotectants that are acetamides, cyclohexanediols, formamides, polyethylene glycol, glycerol, disaccharides and propanediol.

Other cryoprotectants that may be used are described in U.S. Pat. No. 6,395,467 to Fahy et al.; U.S. Pat. No. 6,274,303 to Wowk et al.; U.S. Pat. No. 6,194,137 to Khirabadi et al.; U.S. Pat. No. 6,187,529 to Fahy et al.; U.S. Pat. No. 5,962,214 to Fahy et al.; U.S. Pat. No. 5,955,448 to Calaco et al.; U.S. Pat. No. 5,629,145 to Meryman; and/or WO 02/32225 A2, which corresponds to U.S. Pat. No. 6,740,484 to Khirabadi et al. the enclosures of which are incorporated by reference in their entireties.

The volume of the solutions employed may vary considerably, such as from about 1 to about 100 mls or greater, based upon the size of the piece of tissue being preserved, or the size of the tissue being immersed in solution.

In embodiments, the solution includes cryoprotectants in an aqueous solution, such as Euro-Collins solution, sterile water, salt solutions, culture media, and any physiological solution. Euro-Collins solution (EC-Solution) is an aqueous solution described in Table 2 below.

TABLE 2
Euro-Collins*

| Compound | mM | g/l |
|---|---|---|
| Dextrose | 194 | 34.96 |
| KH₂PO₄ | 15 | 2.06 |
| K₂HPO₄ | 42 | 7.40 |
| KCl | 15 | 1.12 |
| NaHCO₃ | 10 | 0.84 |

*pH = 7.4
*milliosmolality = 350-365 milliosmolal

Other examples of suitable aqueous solutions are discussed in Tables 3 and 4 below.

TABLE 3
Modified RPS-2

| Compound | mM | g/l |
|---|---|---|
| Dextrose | 180 | 32.43 |
| K₂HPO₄ | 7.2 | 1.25 |
| KCl | 28.2 | 2.11 |
| NaHCO₃ | 10 | 0.84 |
| Glutathione | 5 | 1.53 |
| Adenine HCl | 1 | 0.17 |
| CaCl₂ | 1 | 0.111 |
| MgCl₂ | 2 | 0.407 |

Note:
(RPS-2 ™ solution is modified RPS-2 without CaC1₂ and also without MgCl₂)

TABLE 4

| Modified UW Solution #1 | | | Modified UW Solution #2 | | |
|---|---|---|---|---|---|
| Compound | mM | g/l | Compound | mM | g/l |
| NaH₂PO₄•H₂O | 25 | 3.45 | NaH₂PO₄•H₂O | 25 | 3.45 |
| K gluconate | 100 | 23.42 | K gluconate | 100 | 23.42 |
| Mg gluconate | 1 | 0.21 | Mg gluconate | 1 | 0.21 |
| glucose | 5 | 0.90 | Glucose | 15 | 2.70 |
| glutathione | 3 | 0.92 | Glutathione | 3 | 0.92 |
| adenosine | 5 | 1.34 | Adenosine | 5 | 1.34 |
| HEPES | 10 | 2.38 | HEPES | 10 | 2.38 |
| adenine | 1 | 0.17 | adenine | 1 | 0.17 |
| (hydrochloride) | | | (hydrochloride) | | |
| Ribose | 1 | 0.15 | Ribose | 1 | 0.15 |
| CaCl₂ | 0.05 | 0.0056 | CaCl₂ | 0.05 | 0.0056 |
| HES(g) | — | 50 | — | — | — |

Note:
(Modified UW Solution #2 does not contain HES but contains more glucose than modified UW Solution #1)

The vehicle for the cryoprotectant solution may be any type of solution that maintains matrix integrity under in vitro conditions. In embodiments, the vehicle generally comprises slowly penetrating solutes. In embodiments, the vehicle solution is a Euro-Collins solution containing 10 mM HEPES. HEPES is included as a buffer and may be included in any effective amount. In addition, other buffers, as well as no buffer, may be used. Alternative vehicles include, for example, the solutions discussed in Tables 2 and 3 above.

The final concentration of the cryoprotectant solution used for tissue preservation is at least 75% cryoprotectant by weight. In embodiments, the tissue to be preserved, such as a cryoprotectant-free tissue, or a substantially cryoprotectant-free tissue, which may or may not have been previously exposed to a cryoprotectant, may be immersed in (or exposed to) a single solution having a cryoprotectant concentration of at least 75% (by weight) in a single step. In embodiments, such a single step may increase the concentration of the cryoprotectant in the solution in which the tissue is immersed from less than 1M to greater than 12M, increasing the concentration of the cryoprotectant in the solution in which the tissue is immersed from less than 0.5M to greater than 15M. In embodiments, such a single step may kill a majority of the living cells present or all living cells present (e.g., by exposure to the extreme conditions, such as severe osmotic stresses and/or chemical cytotoxicity). In embodiments, the tissue may be immersed in solution having a cryoprotectant concentration of at least 75% (by weight) for a time sufficient for the cryoprotectant to permeate the tissue, such as at least 15 minutes, or at least 60 minutes, or at least 120 minutes.

After the tissue has been immersed in a solution containing a concentration of cryoprotectant sufficient to reach the desired concentration of at least 75% by weight cryoprotectant, the tissue, which is maintained in a solution containing a concentration of cryoprotectant of at least 75% by weight cryoprotectant, may be cooled and stored at any refrigerated temperature up to and including room temperature. In some embodiments, the tissue, which is maintained in a solution containing a concentration of cryoprotectant of at least 75% by weight cryoprotectant, may be cooled and stored at a temperature between −20° C. and below the solutions glass transition temperature. The cooling rate may be from about −0.5 to about −100° C. per minute.

In embodiments, cooling rates (for single or multi-step cooling processes) include, for example, cooling rates in the range from about 0.5 to about 10° C./min, such as about 2 to about 8° C./min, or about 4 to about 6° C./min. In embodiments, the process is independent of cooling rate as long as ice formation is avoided.

The tissue may be stored for a period of time at the desired temperature without cracking and ice formation.

After storage the tissue may be removed from the at least 75% by weight cryoprotectant solution with or without perfusion. Methods for removing the tissue from the at least 75% by weight cryoprotectant solution may comprise slowly warming the tissue in the at least 75% by weight cryoprotectant solution to a warmer temperature. A slow warming rate below 50° C. per minute may be used to warm the tissue in the at least 75% by weight cryoprotectant solution. In embodiments, the average warming rate during this stage may be from about 10-40° C. per minute, such as from about 25-35° C. per minute.

After the tissue has undergone this warming process, the tissue may then be warmed to any desired temperature sufficiently high that the solution is sufficiently fluid that the tissue may be removed therefrom. The warming process may be conducted at any desired rate. In embodiments, the tissue may be warmed to a temperature above about −20° C., such as above about −10° C., or to a temperature above about −5° C., such as between about −5° C. and about 5° C. In embodiments, the process is independent of warming rate as long as ice formation is avoided.

In embodiments, the warming rate may be achieved by changing the environment in which the container (e.g., sterile polyester bags or vials) containing the solution is placed. In embodiments, the slow warming rate may be achieved by placing the container (e.g., sterile polyester bags or vials) in a gaseous environment at a temperature above the temperature at which the tissue has been stored. Then, to achieve the rapid warming rate, the container (e.g., sterile polyester bags or vials) may be placed in a liquid, such as an aqueous solution of, for example, dimethyl sulfoxide (DMSO), at a temperature above −75° C., such as above 0° C., or at normal atmospheric temperatures.

In embodiments, after the tissue has been warmed to a temperature above −65° C., the concentration of the cryoprotectant in the solution may be reduced in a single, gradient, or stepwise manner. In embodiments, the tissue (such as a tissue that has been immersed in the least 75% by weight cryoprotectant solution) in which the concentration of the cryoprotectant is to be reduced may be immersed in (or exposed to) a cryoprotectant-free solution or substantially cryoprotectant-free solution in a single step. In embodiments, such a single step may decrease the concentration of the cryoprotectant in the initial solution and form a substantially cryoprotectant-free solution; for example, the concentration of the solution in which the tissue is immersed may be decreased from greater than 12M to less than 1M in a single step (or multiple steps), such as from greater than 15M to less than 0.1M in a single step (or multiple steps). In embodiments, the tissue may be immersed in the cryoprotectant-free solution or substantially cryoprotectant-free solution for a time sufficient for the cryoprotectant to exit the tissue, such as at least 15 minutes, or at least 60 minutes, or at least 120 minutes.

In embodiments, the tissue in which the cryoprotectant concentration is to be reduced may be immersed in (or exposed to) a solution in which the cryoprotectant concentration of the solution is may be gradually decreased, such as a by use of a linear or nonlinear concentration gradient, to achieve a substantially cryoprotectant-free solution or cryoprotectant-free solution. In embodiments, the concentration gradient is a linear or nonlinear concentration gradient in which a solution having a cryoprotectant concentration of at least 75% by weight is gradually replaced with a cryoprotectant-free solution. For example, the solution having a cryoprotectant concentration of at least 75% by weight may be substantially (at least 99% by weight) replaced by a cryoprotectant-free solution in less than about 30 minutes, such as less than about 10 minutes, or less than about 5 minutes, or less than about 1 minute. In embodiments, the change in concentration in the gradient process is slow enough to achieve approximate osmotic equilibration.

In embodiments, the cryoprotectant concentration is reduced in a step-wise manner. In embodiments, decreasing the cryoprotectant concentration of the tissue may be achieved by immersing the tissue in a series of decreasing cryoprotectant concentration solutions to facilitate elution of cryoprotectants from the tissue. The tissue may also be perfused with the solutions. The solutions are generally at a temperature above about −15° C., such as between about −15° C. and about 37° C., or between about 0° C. and about 25° C.

In embodiments, the cryoprotectant concentration may be reduced to achieve a particular plateau, which may be maintained for a sufficient time to achieve approximate osmotic equilibration, for example for at least about 10 minutes, such as for about 15 minutes. Then, the cryoprotectant concentration may be further reduced, which may or may not provide for a cryoprotectant-free solution. If not, after maintaining the concentration for sufficient time to achieve approximate osmotic equilibration, the cryoprotectant concentration may be again further reduced in one or more steps to eventually provide a cryoprotectant-free solution. In embodiments, the tissue may be immersed in each solution for at least 15 minutes, or longer than an hour.

To decrease the cryoprotectant concentration, the cryoprotectant solution may be mixed with a solution of a type similar to the cryoprotectant-free solution utilized in adding cryoprotectant to the tissue. The solution may also comprise at least one osmotic buffering agent.

As used herein, “osmotic buffering agent” means a low or high molecular weight non-penetrating extracellular solute that counteracts the osmotic effects of the greater intracellular than extracellular concentrations of cryoprotectant during the cryoprotectant efflux process. As used herein, the term “non-penetrating” means that the great majority of molecules of such chemicals do not penetrate into the cells of the tissue but instead remain in the extracellular fluid of the tissue.

As used herein, “low molecular weight” refers, for example, to a relative molecular mass of 1,000 daltons or less. As used herein, “low molecular weight osmotic buffering agents” have a relative molecular mass of 1,000 daltons or less. Low molecular weight osmotic buffering agents include, for example, maltose, potassium and sodium fructose 1,6-diphosphate, potassium and sodium lactobionate, potassium and sodium glycerophosphate, maltopentose, stachyose, mannitol, sucrose, glucose, maltotriose, sodium and potassium gluconate, sodium and potassium glucose 6-phosphate, and raffinose. In embodiments, the low molecular weight osmotic buffering agent is at least one of mannitol, sucrose and raffinose.

As used herein, “high molecular weight” refers, for example, to a relative molecular mass of from greater than 1,000 to 500,000 daltons. As used herein, “high molecular weight cryoprotectant and osmotic buffering agents” generally have a relative molecular mass of from greater than 1,000 to 500,000 daltons. High molecular weight osmotic buffering agents include, for example, hydroxyethyl starch (HES), polyvinylpyrrolidone (PVP), potassium raffinose undecaacetate (>1,000 daltons) and Ficoll (greater than 1,000 to 100,000 daltons). In embodiments, the high molecular weight osmotic buffering agent is HES, such as HES having a molecular weight of about 450,000.

The cryoprotectant-free solution may contain less than about 500 mM of an osmotic buffering agent, such as from about 200 to 400 mM osmotic buffering agent. As the osmotic buffering agent, a low molecular weight osmotic buffering agent may be used. In embodiments, the low molecular weight osmotic buffering agent is mannitol.

In embodiments, the cryoprotectant may be removed in a series of steps such as three, four, five, six, seven, etc. steps. In embodiments, the cryoprotectant may be removed in a series of seven steps, where in step 1, the tissue may be exposed to a cryoprotectant solution with a concentration that may be about 40 to about 80%, such as about 55 to about 75%, of the highest cryoprotectant concentration used; in a step 2, the tissue may be exposed to a cryoprotectant concentration that may be about 30 to about 45%, such as about 35 to about 40%, of the highest cryoprotectant concentration used; in step 3, the tissue may be exposed to a cryoprotectant concentration that may be about 15 to about 35%, such as about 20 to about 30%, of the highest cryoprotectant concentration used; in step 4, the tissue may be exposed to a cryoprotectant concentration that may be about 5 to about 20%, such as about 10 to about 15%, of the cryoprotectant concentration used; and in step 5, the tissue may be exposed to a cryoprotectant concentration that may be about 2.5 to about 10%, such as about 5 to about 7.5%, of the cryoprotectant concentration used. In the above steps, the remainder of the solution may be cryoprotectant-free solution containing osmotic buffering agent. In step 6, essentially all of the cryoprotectant may be removed and the osmotic buffering agent may be retained. In step 7, the osmotic buffering agent may be removed. In embodiments, steps 6 and 7 may be combined in a single step. For example, the osmotic buffering agent may be removed at the same time as the remainder of the cryoprotectant. In embodiments, if no osmotic buffering agent is used or if it is not removed, step 7 can be eliminated. Each of these concentration steps may be maintained for a sufficient time to achieve approximate osmotic equilibration, such as about 10 to 30 minutes, or 15 to 25 minutes. In embodiments, the cryoprotectant is removed in one or more washes employing a cryoprotectant-free solution.

The temperature of the series of solutions used for removing the cryoprotectant from the tissue may be above about −15° C., such as between about −15 and about 15° C., or between about 0° C. and about 37° C. or higher provided that the tissue is not exposed to denaturation conditions. In embodiments, step 1 may be started when the tissue is at a temperature above about −75° C., such as above −65° C. In embodiments, the temperature of the tissue may be below the temperature of the solution in which it is immersed in step 1, and the tissue may be further warmed to a temperature above about −15° C. during step 1 of the cryoprotectant removal.

The cryoprotectant-free solution employed for washing of the tissue may be sterile water, a physiological salt solution (for example saline, Hank's Balanced Salt Solution, Lactated Ringers Solution or Krebs-Henseliet Solution) or tissue culture media (for example Roswell Park Memorial Institute media, Dulbecco's Modified Eagle's Medium (DMEM), Eagle's Medium or Medium 199) employed for tissues, such as mammalian cells.

The number of washes, volume of each wash and duration of each wash may vary depending upon the tissue mass and the final residual chemical concentrations desired. In embodiments, the last wash (rinse) may be in a commonly employed medical salt solution, such as saline or Ringers Solution.

The tissue may be further processed after storage. For example, after storage the tissues may be seeded with patient cells. Thus, these ice-free preserved tissues may provide materials for the manufacturing of more complex tissue engineered implants for medical applications.

In a first aspect, the present disclosure relates to a method for preserving a tissue and reducing the immune reaction after transplantation or implantation of the preserved tissue, comprising: obtaining a first tissue from a donor, the first tissue being a wild type tissue or genetically modified tissue; forming a second tissue by immersing the first tissue in a first solution having a cryoprotectant concentration of at least about 75% by weight for at least one hour to kill and lyse the cells of the first tissue; forming a third tissue by removing residual cell materials of the second tissue, the residual cell materials of the second tissue being removed by subjecting the second tissue to decellularization in a bioreactor; subjecting the third tissue to ice-free cryopreservation, the ice-free cryopreservation including: infiltrating the third tissue with a second solution having a cryoprotectant concentration of at least about 75% by weight by placing the third tissue and the second solution in a container at a predetermined temperature for at least one hour, removing the second solution and replacing it with a third solution having a cryoprotectant concentration of at least about 75% by weight, sealing the container after the second solution has been replaced with the third solution such that the sealed container contains the third solution and third tissue, and storing the sealed container; and implanting or transplanting the third tissue into a recipient, wherein an immune reaction is not precipitated by the transplantation or implantation of the third tissue, or any immune reaction that occurs in the recipient is non-life-threating. In a second aspect, the present disclosure further relates to the method of the first aspect, wherein the origin of the first tissue is a porcine source that has been genetically engineered. In a third aspect, the present disclosure further relates to the method of any of the preceding aspects, wherein the wild type tissue or genetically modified tissue is selected from the group consisting of heart valves, pericardium, blood vessels, ligaments, tendons, bladder, intestine, and skin. In a fourth aspect, the present disclosure further relates to the method of any of the preceding aspects, wherein the first tissue, the second tissue, and third tissue are not crosslinked with glutaraldehyde. In a fifth aspect, the present disclosure further relates to the method of any of the preceding aspects, wherein the second tissue is formed in one step by placing the first tissue in sterile packaging at room temperature on a shaker in 10 to 80 mL of the first solution for a duration that is sufficient to kill all of the living cells of the first tissue. In a sixth aspect, the present disclosure further relates to the method of any of the preceding aspects, wherein the third tissues are formed from the second tissues in 1 to 5 days or a period that is longer than five days, the residual cell materials of the second tissue are removed by washing in sterile bioreactors under physiologic flow and pressure conditions with a sterile solution. In a seventh aspect, the present disclosure further relates to the method of any of the preceding aspects, wherein the ice-free cryopreservation comprises placing the third tissue and second solution in a sterile polyester bag or sterile polyester vial on a shaker for at least one hour at room temperature. In an eighth aspect, the present disclosure further relates to the method of any of the preceding aspects, where the ice-free cryopreservation comprises placing the third tissue and second solution in a sterile polyester bag at room temperature on a shaker for at least one hour, and then, after the second solution replaced with the third solution, the polyester bag is heat sealed and stored. In a ninth aspect, the present disclosure further relates to the method of any of the preceding aspects, wherein the first tissue, the second tissue, and the third tissue are each a tissue where a galactose-α(1,3)-galactose antigen (α-Gal) epitope is not present. In a tenth aspect, the present disclosure further relates to the method of any of the preceding aspects, wherein the third tissue is formed via detergent-free decellularization in a dynamic flow bioreactor. In an eleventh aspect, the present disclosure further relates to the method of any of the preceding aspects, wherein the third tissue is 99% DNA free. In an twelfth aspect, the present disclosure further relates to the method of any of the preceding aspects, method of any of the preceding claims, wherein the cryoprotectant comprises at least one molecule selected from the group consisting of acetamides, cyclohexanediols, formamides, dimethyl sulfoxide, ethylene glycol, polyethylene glycol, glycerol, disaccharides and propanediol. In thirteenth aspect, the present disclosure further relates to the method of any of the preceding aspects, wherein said cryoprotectant solution comprises at least one member selected from the group consisting of acetamide, agarose, alginate, alanine, albumin, ammonium acetate, anti-freeze proteins, butanediol, chondroitin sulfate, chloroform, choline, cyclohexanediols, dextrans, diethylene glycol, dimethyl acetamide, dimethyl formamide, dimethyl sulfoxide, erythritol, ethanol, ethylene glycol, ethylene glycol monomethyl ether, formamide, glucose, glycerol, glycerophosphate, glyceryl monoacetate, glycine, glycoproteins, hydroxyethyl starch, inositol, lactose, magnesium chloride, magnesium, sulfate, maltose, mannitol, mannose, methanol, methoxy propanediol, methyl acetamide, methyl formamide, methyl ureas, methyl glucose, methyl glycerol, phenol, pluronic polyols, polyethylene glycol, polyvinylpyrrolidone, proline, 1,2-propanediol, pyridine N-oxide, raffinose, ribose, serine, sodium bromide, sodium chloride, sodium iodide, sodium nitrate, sodium nitrite, sodium sulfate, sorbitol, sucrose, trehalose, triethylene glycol, trimethylamine acetate, urea, valine and xylose. In a fourteenth aspect, the present disclosure further relates to the method of any of the first eleven aspects, wherein the first solution, the second solution and the third solution are each a 83% cryoprotectant solution containing 4.65 M DMSO, 4.65 M formamide, and 3.31 M 1,2 propanediol in Euro-Collins solution. In a fifteenth aspect, the present disclosure further relates to the method of any of the preceding aspects, wherein the wild type tissue or genetically modified tissue is a heart valve. In a sixteenth aspect, the present disclosure further relates to the method of any of the preceding aspects, wherein the wild type tissue or genetically modified tissue is a pulmonary valve. In a seventeenth aspect, the present disclosure further relates to the method of any of the preceding aspects, wherein the wild type tissue or genetically modified tissue is an artery. In an eighteenth aspect, the present disclosure further relates to the method of any of the preceding aspects, wherein the sealed container is stored at a controlled temperature. In a nineteenth aspect, the present disclosure further relates to the method of any of the preceding aspects, wherein the sealed container is stored at room temperature. In a twentieth aspect, the present disclosure further relates to the method of any of the preceding aspects, wherein the sealed container is stored at a temperature between about +40° C. and below the glass transition temperature of the third solution. In a twenty-first aspect, the present disclosure further relates to the method of any of the preceding aspects, wherein the third tissue has reduced immunogenicity in humans as compared to a corresponding wild type tissue or genetically modified tissue obtained from the same donor, the corresponding wild type tissue or genetically modified tissue obtained from the same donor being either: a tissue that was subject to only decelluarization or cryoprotectant exposure, a tissue having reduced immunogenicity that was achieved by via hiding or masking antigens, a tissue that was not subject to decellularization and/or cryoprotectant exposure, or an unmodified tissue.

In a twenty-second aspect, the present disclosure further relates to the method of any of the preceding aspects, the third tissue has reduced immunogenicity in humans as compared to a corresponding wild type tissue or genetically modified tissue obtained from the same donor, the corresponding wild type tissue or genetically modified tissue obtained from the same donor having reduced immunogenicity was achieved by via crosslinking with glutaraldehyde. In a twenty-third aspect, the present disclosure further relates to a method for reducing tissue immunogenicity, comprising: obtaining a first tissue from a donor, the first tissue being a wild type tissue or genetically modified tissue; forming a second tissue by immersing the first tissue in a first solution having a cryoprotectant concentration of at least about 75% by weight for at least one hour to kill and lyse the cells of the first tissue; forming a third tissue by removing residual cell materials of the second tissue, the residual cell materials of the second tissue being removed by subjecting the second tissue to decellularization in a bioreactor; wherein after being transplanted for a predetermined time, such as 1 day, one week, or one year, the third tissue stimulates less of an immune response in humans as compared to a corresponding wild type tissue or genetically modified tissue obtained from the same donor, the corresponding wild type tissue or genetically modified tissue obtained from the same donor being either: a tissue that was subject to only decelluarization or cryoprotectant exposure, a tissue having reduced immunogenicity that was achieved by via hiding or masking antigens, a tissue that was not subject to decellularization and/or cryoprotectant exposure, or an unmodified tissue.
In a twenty-fourth aspect, the present disclosure further relates to the method of the preceding aspect, wherein the immune response is assessed in terms of an inflammatory mediator concentration, the inflammatory mediator being one or more member selected from the group consisting of: cytokines, histamine, bradykinin, prostaglandins, and leukotrienes; and the immune response in humans to the third tissue results in an inflammatory mediator concentration that is either: no greater than ⅓ of that of the corresponding wild type tissue or genetically modified tissue obtained from the same donor, no greater than 1/10^{th} that that of the corresponding wild type tissue or genetically modified tissue obtained from the same donor, or at least two orders of magnitude below that of the corresponding wild type tissue or genetically modified tissue obtained from the same donor. In a twenty-fifth aspect, the present disclosure further relates a method for preserving a tissue, comprising: obtaining a first tissue from a donor, the first tissue being a wild type tissue or genetically modified tissue; forming a second tissue by exposing cells of the first tissue to a cryoprotectant concentration that is sufficient to kill and lyse the cells of the first tissue; forming a third tissue by removing residual cell materials of the second tissue, the residual cell materials of the second tissue being removed by subjecting the second tissue to bioreactor-mediated decellularization; and subjecting the third tissue to ice-free cryopreservation, the ice-free cryopreservation including: infiltrating the third tissue with a second solution having a cryoprotectant concentration of at least about 75% by weight by placing the third tissue and the second solution in a container at a predetermined temperature for at least one hour, removing the second solution and replacing it with a third solution having a cryoprotectant concentration of at least about 75% by weight, sealing the container after the second solution has been replaced with the third solution such that the sealed container contains the third solution and third tissue, and storing the sealed container. In a twenty-sixth aspect, the present disclosure further relates to a method for preserving a tissue, comprising: obtaining a first tissue from a donor, the first tissue being wild type tissue or genetically modified tissue; forming a second tissue by exposing cells of the first tissue to a cryoprotectant concentration that is sufficient to kill and lyse the cells of the first tissue; forming a third tissue by removing residual cell materials of the second tissue, the residual cell materials of the second tissue being removed by subjecting the second tissue to bioreactor-mediated decellularization; and placing the third tissue and the second solution in a container, and storing the sealed container; wherein the third tissue is not subjected to ice-free cryopreservation prior to being transplanted. In a twenty-seventh aspect, the present disclosure further relates to the method of any of the preceding aspects, A method for preserving a tissue, comprising: obtaining a first tissue, the first tissue being a wild type tissue or genetically modified tissue; forming a second tissue by immersing the first tissue in a first solution having a cryoprotectant concentration of at least about 75% by weight for at least one hour to kill and lyse the cells of the first tissue; forming a third tissue by removing residual cell materials of the second tissue, the residual cell materials of the second tissue being removed by subjecting the second tissue to decellularization in a bioreactor; and subjecting the third tissue to ice-free cryopreservation, the ice-free cryopreservation including: infiltrating the third tissue with a second solution having a cryoprotectant concentration of at least about 75% by weight by placing the third tissue and the second solution in a container at a predetermined temperature for at least one hour, removing the second solution and replacing it with a third solution having a cryoprotectant concentration of at least about 75% by weight, sealing the container after the second solution has been replaced with the third solution such that the sealed container contains the third solution and third tissue, and storing the sealed container. In a twenty-eighth aspect, the present disclosure further relates to the method of the preceding aspect, wherein the origin of the first tissue is a porcine source that has been genetically engineered. In a thirtieth aspect, the present disclosure further relates to the method of the twenty-seventh aspect and/or twenty-eighth aspect, wherein the wild type tissue or genetically modified tissue is selected from the group consisting of heart valves, pericardium, blood vessels, ligaments, tendons, bladder, intestine, and skin. In a twenty-ninth aspect, the present disclosure further relates to the method of the twenty-seventh aspect and/or twenty-ninth aspect, wherein the first tissue, the second tissue, and third tissue are not crosslinked with glutaraldehyde. In a thirty-first aspect, the present disclosure further relates to the method of any of the preceding aspects from the twenty-seventh aspect to the thirtieth aspect, wherein the second tissue is formed in one step by placing the first tissue in sterile packaging at room temperature on a shaker in 10 to 80 mL of the first solution for a duration that is sufficient to kill all of the living cells of the first tissue. In a thirty-second aspect, the present disclosure further relates to the method of any of the preceding aspects from the twenty-seventh aspect to the thirty-first aspect, wherein the third tissues are formed from the second tissues in 1 to 5 days or a period that is longer than five days, the residual cell materials of the second tissue are removed by washing in sterile bioreactors under physiologic flow and pressure conditions with a sterile solution. In a thirty-third aspect, the present disclosure further relates to the method of any of the preceding aspects from the twenty-seventh aspect to the thirty-first aspect, wherein the ice-free cryopreservation comprises placing the third tissue and second solution in a sterile polyester bag or sterile polyester vial on a shaker for at least one hour at room temperature. The foregoing is further illustrated by reference to the following examples, which are presented for purposes of illustration and are not intended to limit the scope of the present disclosure.

### Examples

Tests were performed to determine the inflammatory response, remodeling and immunogenicity of porcine tissues post-VS83 cryopreservation. Fresh (F-WT and F-KO) and IFC tissues (V-WT and V-KO) were compared by subcutaneous transplantation in GalSafe® α-Gal knockout pigs. Ten GalSafe® pigs derived from four separate litters were delivered to the Medical University of South Carolina (MUSC) under the care and direction of Dr. Brockbank and Dr. Kris Helke, DVM, PhD. Prior to shipment, blood was collected from each pig for confirmation of genotype and phenotype. Genotypic confirmation was performed via LR-PCR for the presence of the genetic insert on both alleles of GGTA1. Control and treated tissues (aorta and tendon) were implanted subcutaneously along the back over muscle in 8 GalSafe® pigs under anesthesia, 2 GalSafe® pigs and 2 WT pigs provided tissue samples. The implants were retrieved with the surrounding tissues after 2 or 4 weeks. The explants were cut in half and formalin fixed. The tissues were rinsed and placed in buffer solution after 24 hours of fixation. The tissues were sent to a service lab for embedding, sectioning, and H&E staining Semi-quantitative blind histopathology review of the explant also demonstrated reduced inflammatory cell infiltration in WT and GalSafe® ice-free vitrified tendon and aorta explants with many significant differences compared with fresh untreated controls (The results are illustrated in FIG. 1).

Further tests will also be performed using WT porcine pulmonary HVs from a local food processing plant. Confirmatory experiments will be performed with GalSafe® tissues obtained from Revivicor's service provider for pig breeding and maintenance. Two in vitro test systems will be employed. In the first test system, conditioned media obtained from 10% DMSO cryopreserved tissue punches will be treated with increasing doses of propanediol, formamide and DMSO in EuroCollins solution up to 50% (v/v). The conditioned media will be characterized for different activated TGF-β isoforms using ELISAs. Activated TGF-β isoform release will be expressed in % of the latent TGF-β in frozen (CFC) conditioned medium. This test system will define the concentrations of each CPA that activate TGF-β isoform release and in what combinations. In the second test system, tissue punches will be cryopreserved with the experimental VS83 CPA formulation+decellularization and compared with untreated fresh and 10% DMSO frozen arterial punches as negative controls and VS83 treated punches. Cytokines including latent and active TGF-β isoforms will be assessed in conditioned media derived from experimental and control tissues. The inventors have previously demonstrated that VS83 treated porcine tissue (without decellularization) has similar outcomes as VS83 treatment of human tissue resulting in decreased hPBMC proliferation including T-memory cells (Seifert, 2015).

Comparison of outcomes using the two test systems are anticipated to indicate the concentrations and combinations of the three CPAs in VS83 that will potentially impact the immune response. In contrast to both TGF-β1 and β2 isoforms, TGFβ-3 mediates anti-fibrotic effects that could impact tissue remodeling. Therefore, testing will be for both TGF-β3 and -β2 isoforms as well as TGF-β1. TGF-β isoforms are highly conserved across mammalian species. The test systems used are expected to work because cDNA cloning has shown total sequence identity between the respective human and porcine sequences (p'Sporn, 1987). Dose dependent activation of TGF-β1 is anticipated by formamide and DMSO using porcine tissue, confirming earlier results with human tissue. Demonstration that porcine tissue cytokine release is similar to human (Schneider, 2017) will confirm that similar mechanisms of action are operating in VS83 treated porcine tissues.

It is anticipated that that α-Gal Knockout heart valves that have been decellularized and IFC by vitrification (α-Gal Knockout Decell+V) will have minimal if any structural deterioration or decrease in in vivo function. Similar outcomes are expected for α-Gal Knockout-Decell stored in sterile PBS because the valves will be exposed to CPAs prior to decellularization. It is considered unlikely that the WT Decell+V group will perform well due to α-Gal associated with the valve ECM. Similarly, we anticipate failure of the WT-Decell alone due to presence of ECM associate α-Gal. It is unlikely that additional engineering of the GalSafe® genome is needed for tissue scaffolds.

Further genetic enhancements under consideration for xenotransplantation are additional gene knockouts such as N-glycolylneuraminic acid (Neu5Gc). α-Gal is on cell surfaces and within most mammalian tissue matrices while Neu5Gc appears to be limited to cell surfaces. Neu5Gc is not involved in the etiology of AGS (Apostolovic, 2014). Unlike α-Gal, Neu5Gc is present in cells of some human subjects. α-Gal antibodies are detected in all humans at high levels, 1-3% of circulating IgG, however, corresponding levels of anti Neu5GC antibodies are quite variable, and are not universally detectable. Anti-Neu5Gc antibodies, when present, are <0.1% of circulating IgG. Therefore it is unlikely that further genetic modification is needed.

Procurement of Heart Valves: WT hearts for the experiments will be obtained from market sized donors (about 4 months of age) at a local food processing plant. Male and female juvenile (WT and Galsafe®) pigs hearts from Revivicor may also be employed. The hearts will be washed in ice-cold Hanks Balanced Salts Solution (HBSS) and placed in HBSS on ice with antibiotics (126 mg/L Lincomycin, 52 mg/L, 10 Vancomycin, 157 mg/l Cefoxitin and 117 mg/L Polymixin) for transport to the dissection lab. After dissection under aseptic conditions in a class 100 biosafety hood the aortic valve internal diameter and anatomy will be recorded and antibiotic treated for 24 hours at 4° C. before further treatment. This step may be avoided by sterile procurement or terminal sterilization using chemical and or irradiation techniques commonly employed in mammalian tissue processing for products destined for implantation, transplantation, in patients.

Bioreactor-mediated decellularization will be conducted as described above in connection with FIG. 1. Ice-free cryopreservation will be performed as previously described (Brockbank, 2015). Tissue will be infiltrated with 83% CPA solution containing 4.65 M DMSO, 4.65 M FMD, and 3.31 M 1,2 propanediol [propylene glycol (PG)] in Euro-Collins (EC) solution in one step by placing the tissues in sterile polyester bags or vials at room temperature on a shaker for at least one hour (10-80 mL of VS83 in EC solution depending upon tissue volume). Then the CPA solution is removed and replaced with fresh VS83 CPA solution, the bag heat sealed, and then rapidly cooled. The cooling process will then be achieved by placing the bags for 10 minutes in a pre-cooled bath of 2-methylbutane (<−100° C.) in either a −135° C. mechanical freezer or in the top of a nitrogen cooled freezer, and then stored at −80° C. After at least one week of storage, tissue will be rapidly rewarmed in a 37° C. water bath and the CPA solution will be removed by placing the tissue in pre-cooled EC-solution containing mannitol, followed by EC-solution alone and finally in 4° C. Dulbecco's Modified Eagle Medium (DMEM) or lactated Ringer's with 5% dextrose (LRD5).

Hemodynamics and Leaflet Mechanics: Fresh and ice-free cryopreserved HVs decellularization will be mounted in the HV bioreactor (FIG. 1) and tested for functionality, for example under aortic conditions we will use 70.5 bpm, 70 mL/stroke, 120/80 mmHg with data capture using a high speed camera (240 fps) and digital imaging. Geometric orifice area (GOA) measurements will be calculated in mm² and plotted as a function of time over multiple cycles (Schleicher, 2010). The mechanical properties of the leaflets will also be assessed using established methods (Brockbank, 2011; Sierad, 2015).

Histology/Immunohistochemistry: Decellularized tissue quality screening for residual cell materials includes DNA extraction/analysis (Cyquant assay) and histology methods. Representative samples from the individual tissue components of the HV explants will be processed and used for qualitative and quantitative morphometric histology review of stained sections. Stains will include Hematoxylin & Eosin (H&E) and elastin stains (Song, 2000), DAPI staining and Movat's pentachrome. Immunostaining will identify residual cell fragments after decellularization using immunohistochemistry.

Measurement of Cytokine production/release as measures of potential immunogenicity: Tissue punches (6 mm diameter, n=8-10) will be incubated in DMEM for 1-7 days at 37° C. Supernatants will be taken and analyzed for cytokine analysis, including latent and active TGF-β isoforms, using an enzyme-linked immunosorbent assay (ELISA) according to manufacturer protocols. Kinetic analysis will be performed by collecting supernatant each day from the same culture well and the cytokine concentration will be back calculated to the remaining volume of the culture medium to obtain the cytokine amount in pg/mg tissue dry weight. Absorbance will be measured at 450 nm on a plate reader.

Measurement of human peripheral blood mononuclear cell (PBMC) reactivity as a measure of immunogenicity: Tissue punches (6 mm diameter, n=8-10) may be co-cultured with human PBMCs to determine whether the proliferative effect of fresh untreated tissue punches is ameliorated using methods described by Seifert (2015).

All literature and patent references cited throughout the disclosure are incorporated by reference in their entireties.

What is claimed is:

1. A method for preserving a tissue and reducing the immune reaction after transplantation or implantation of the preserved tissue, comprising:
obtaining a first tissue from a donor, the first tissue being a wild type tissue or genetically modified tissue;
forming a second tissue by immersing the first tissue in a first solution having a cryoprotectant concentration of at least about 75% by weight for at least one hour to kill and lyse the cells of the first tissue;
forming a third tissue by removing residual cell materials of the second tissue, the residual cell materials of the second tissue being removed by subjecting the second tissue to decellularization in a bioreactor;
subjecting the third tissue to ice-free cryopreservation, the ice-free cryopreservation including:
infiltrating the third tissue with a second solution having a cryoprotectant concentration of at least about 75% by weight by placing the third tissue and the second solution in a container at a predetermined temperature for at least one hour,
removing the second solution and replacing it with a third solution having a cryoprotectant concentration of at least about 75% by weight, sealing the container after the second solution has been replaced with the third solution such that the sealed container contains the third solution and third tissue, and
storing the sealed container; and
implanting or transplanting the third tissue into a recipient, wherein
an immune reaction is not precipitated by the transplantation or implantation of the third tissue, or
any immune reaction that occurs in the recipient is non-life-threating.

2. The method of claim 1, wherein the origin of the first tissue is a porcine source that has been genetically engineered.

3. The method of claim 1, wherein the wild type tissue or genetically modified tissue is selected from the group consisting of heart valves, pericardium, blood vessels, ligaments, tendons, bladder, intestine, and skin.

4. The method of claim 1, wherein the first tissue, the second tissue, and third tissue are not crosslinked with glutaraldehyde.

5. The method of claim 1, wherein the second tissue is formed in one step by placing the first tissue in sterile packaging at room temperature on a shaker in 10 to 80 mL of the first solution for a duration that is sufficient to kill all of the living cells of the first tissue.

6. The method of claim 1, wherein the third tissues are formed from the second tissues in 1 to 5 days or a period that is longer than five days, and the residual cell materials of the second tissue are removed by washing in sterile bioreactors under physiologic flow and pressure conditions with a sterile solution.

7. The method of claim 1, wherein the ice-free cryopreservation comprises placing the third tissue and second solution in a sterile polyester bag at room temperature on a shaker for at least one hour, and then, after the second solution replaced with the third solution, the polyester bag is heat sealed and stored.

8. The method of claim 1, wherein the first tissue, the second tissue, and the third tissue are each a tissue where a galactose-α(1,3)-galactose antigen (α-Gal) epitope is not present.

9. The method of claim 1, wherein the third tissue is formed via detergent-free decellularization in a dynamic flow bioreactor.

10. The method of claim 1, wherein the third tissue is 99% DNA free.

11. The method of claim 1, wherein the first solution, the second solution and the third solution are each a 83% cryoprotectant solution containing 4.65 M DMSO, 4.65 M formamide, and 3.31 M 1,2 propanediol in Euro-Collins solution.

12. The method of claim 1, wherein the wild type tissue or genetically modified tissue is a heart valve.

13. The method of claim 1, wherein the wild type tissue or genetically modified tissue is an artery.

14. The method of claim 1, wherein the sealed container is stored at room temperature.

15. The method of claim 1, wherein the sealed container is stored at a temperature between about +40° C. and below the glass transition temperature of the third solution.

16. The method of claim 1, wherein the third tissue has reduced immunogenicity in humans as compared to a corresponding wild type tissue or genetically modified tissue obtained from the same donor, the corresponding wild type tissue or genetically modified tissue obtained from the same donor being either:
a tissue that was subject to only decelluarization or cryoprotectant exposure,
a tissue having reduced immunogenicity that was achieved by via hiding or masking antigens,
a tissue that was not subject to decellularization and/or cryoprotectant exposure, or
an unmodified tissue.

17. The method of claim 1, wherein the third tissue has reduced immunogenicity in humans as compared to a corresponding wild type tissue or genetically modified tissue obtained from the same donor, the corresponding wild type tissue or genetically modified tissue obtained from the same donor having reduced immunogenicity was achieved by via crosslinking with glutaraldehyde.

18. A method for reducing tissue immunogenicity, comprising:
obtaining a first tissue from a donor, the first tissue being a wild type tissue or genetically modified tissue;
forming a second tissue by immersing the first tissue in a first solution having a cryoprotectant concentration of at least about 75% by weight for at least one hour to kill and lyse the cells of the first tissue;
forming a third tissue by removing residual cell materials of the second tissue, the residual cell materials of the second tissue being removed by subjecting the second tissue to decellularization in a bioreactor; wherein
after being transplanted for a predetermined time, such as 1 day, one week, or one year, the third tissue stimulates less of an immune response in humans as compared to a corresponding wild type tissue or genetically modified tissue obtained from the same donor, the corresponding wild type tissue or genetically modified tissue obtained from the same donor being either:
a tissue that was subject to only decelluarization or cryoprotectant exposure,
a tissue having reduced immunogenicity that was achieved by via hiding or masking antigens,
a tissue that was not subject to decellularization and/or cryoprotectant exposure, or
an unmodified tissue.

19. The method of claim 18, wherein
the immune response is assessed in terms of an inflammatory mediator concentration, the inflammatory mediator being one or more member selected from the group consisting of: cytokines, histamine, bradykinin, prostaglandins, and leukotrienes; and
the immune response in humans to the third tissue results in an inflammatory mediator concentration that is either:
no greater than ⅓ of that of the corresponding wild type tissue or genetically modified tissue obtained from the same donor,
no greater than 1/10^{th} that that of the corresponding wild type tissue or genetically modified tissue obtained from the same donor, or
at least two orders of magnitude below that of the corresponding wild type tissue or genetically modified tissue obtained from the same donor.

20. A method for preserving a tissue, comprising:
obtaining a first tissue, the first tissue being a wild type tissue or genetically modified tissue;
forming a second tissue by immersing the first tissue in a first solution having a cryoprotectant concentration of at least about 75% by weight for at least one hour to kill and lyse the cells of the first tissue;
forming a third tissue by removing residual cell materials of the second tissue, the residual cell materials of the second tissue being removed by subjecting the second tissue to decellularization in a bioreactor; and
subjecting the third tissue to ice-free cryopreservation, the ice-free cryopreservation including:
infiltrating the third tissue with a second solution having a cryoprotectant concentration of at least about 75% by weight by placing the third tissue and the second solution in a container at a predetermined temperature for at least one hour,
removing the second solution and replacing it with a third solution having a cryoprotectant concentration of at least about 75% by weight, sealing the container after the second solution has been replaced with the third solution such that the sealed container contains the third solution and third tissue, and
storing the sealed container.
